(19) United States
(12) Patent Application Publication
(10) Pub. No.: US 2011/0144022 A1
(43) Pub. Date: Jun. 16, 2011

(54) Anti-Angiogenic Peptide

(75) Inventors: Claudio Brigati, Genova (IT); Monica Morini, Genova (IT); Roberto Benelli, Genova (IT); Simona Minghelli, Genova (IT); Douglas M. Noonan, Genova (IT); Raffaella Dell 'Eva, Genova (IT); Adriana Albini, Genova (IT)

(73) Assignee: ISTITUTO NAZIONALE PER LA RICERCA SUL CANCRO, Genova (IT)

(21) Appl. No.: 11/577,202

(22) PCT Filed: Oct. 13, 2005
(86) PCT No.: PCT/EP05/11022

§ 371 Date: Nov. 7, 2007

(30) Foreign Application Priority Data
Oct. 15, 2004 (IT) MI2004A001962

(51) Int. Cl. A61K 38/10; C07K 7/08

(52) U.S. Cl. 514/13.3; 530/328

(57) ABSTRACT

The invention relates to an anti-angiogenic peptide corresponding to a fragment of angiostatin molecule, pharmaceutical compositions containing it and the use thereof in the preventive or therapeutic treatment of diseases involving angiogenesis.

The invention provides an angiostatin peptide having antiangiogenic activity, pharmaceutical compositions containing it and the use thereof in the preventive or therapeutic treatment of diseases involving angiogenesis.

### BACKGROUND OF THE INVENTION

Angiogenesis is a process whereby new vessels are generated in a tissue or organ. In certain conditions, for instance in tumor diffusion, this process proceeds uncontrolled.

Angiostatin (AST) is an antiangiogenic molecule produced as a fragment of a larger molecule, plasminogen, which has not angiostatic properties. AST induces apoptosis in endothelial cells but may act also on other cellular targets (1). Moreover, it can inhibit vessel neoformation in tumors, both primitive and metastatic. In AST treated animals, neither toxic effects nor resistance have been observed.

AST, its sequence variants and DNA-encoding sequences, as well as methods to isolate, purify and synthetically produce it are described in several patents, including U.S. Pat. Nos. 5,861,372; 5,639,725; 5,792,845; 5,885,795; 5,854,205; 5,854,221; 6,024,688. In view of the therapeutic importance of angiostatin, it is particularly important to find peptide fragments able to reproduce or improve its biologic effects.

### DESCRIPTION OF THE INVENTION

In a first embodiment, the invention provides a peptide fragment of AST endowed with anti-angiogenic activity, able to induce IL-12 and to inhibit granulocyte chemotaxis. The peptide is localized in Kringle 3 of angiostatin molecule and is a 13mer, the sequence of which (HNRTPENFPCKNL—SEQ ID NO:1) is identical in human and murine species. In an angiogenesis in vivo assay utilizing matrigel sponges, the peptide proved more active than angiostatin, unlike other peptides corresponding to different regions of the protein. The activities of the peptide and of angiostatin, but not that of the other peptides tested, were inhibited by and antibody anti IL-12. Ematoxylin-eosin stain confirmed a potent angiogensis inhibition as indicated by hemoglobin quantification. The addition of peptide SEQ ID NO: 1 drastically reduced the local cellular infiltration, thus preventing neovascularization.

In a chemotaxis assay for neutrophils in which CXCR1 and CXCR2-agonist and IL8 were used as chemoattractants, the peptide SEQ ID NO:1 showed an activity significantly higher compared to other angiostatin peptides. Moreover, in a transplantable tumor model for Kaposi sarcoma (KS) cells in nude mice, the peptide was able to diminish tumor growth with respect to angiostatin, after a single administration three days before cell injection. This result is particularly important, considering that angiostatin needs a continuous administration to be therapeutically effective.

In a further aspect, the invention relates to pharmaceutical compositions containing the peptide SEQ ID NO:1. The latter can be administered by the oral, rectal ophthalmic, nasal, topic, intrauterin, vaginal or parenteral (e.g. s.c., i.v., i.m.) routes. Suitable formulations for peptide administration can be solid, e.g. capsules pills or granules; liquid, such as solutions, suspensions, syrups, drops, tinctures, spray or aerosols; or semisolid, such as creams, ointments or gels. The formulations may contain, besides the peptide SEQ ID NO:1, one or more adjuvants, such as Freund's, to enhance the immune response.

The dose of peptide SEQ ID NO:1 depends on the severity of the disease or dysfunction to be treated and on other variables such as age, weight of the subject or routes of administration. For the treatment of animals or humans, a quantity of peptide ranging between 0.1 and 250 mg/kg can be used.

The peptide and its formulations according to the invention are indicated for the therapeutic or preventive treatment of diseases or dysfunctions involving or mediated by angiogenesis, particularly tumors, e.g. solid tumours or leukaemia; ocular diseases, including retinopathy, glaucoma, macular degeneration, corneal rejection, retro-lenticular fibroplasia and rubeosis; vascular diseases such as cardiovascular, acute or chronic inflammatory diseases, including diabetes; organ and tissue degenerative diseases, such as rheumatoid arthritis and atherosclerosis; transplants, especially of ocular or skin tissues; cerebral vascular pathologies.

Compared to angiostatin, the peptide of the invention is more stable, has a higher bioavailability and a better tissue distribution; in addition, it can be easily produced with high purity and at low cost.

The peptide SEQ ID NO:1 can be synthetically prepared according to established procedures (Stuart and Young, 1984, solid phase peptide synthesis, 2^{nd} ed., Pierce Chemical Co.; Tam et al., Am Soc., 1983 105: 6442; Merrifield, 1979, The Gross and Meihofer eds NY Academic Press, 1-284), in solution, solid phase or using an automated synthesizer. Alternatively, the peptide may be produced by recombinant DNA techniques (Sambrook et al., Molecular Cloning, a laboratory manual, CSH Press, CSH, NY, 1982 or Ausbel et al., Current Protocols in Molecular Biology, John Wiley and Sons, Inc. NY, 1987). One or more amino acids within SEQ ID NO:1 may be substituted by different residues in D or L configuration, or chemically modified, for instance by amidation of the carboxy-terminus, linkage to lipophylic groups (i.e. fatty acid residues) glycosylation or conjugation to other molecules, so as to improve the peptide activity profile or bioavailability.

The invention also provides a nucleic acid molecule coding for the peptide SEQ ID NO:1, expression vectors thereof and eukaryotic or prokaryotic cellular hosts containing them.

Materials and Methods

1. Xenotransplant of Tumor Cells

CD1 nude mice were injected in the flank with 5 million KS-imm cells and split in four groups: one was injected peri-tumorally with 2.5 μg AST (Calbiochem, La Jolla, Calif.) in 100 μl PBS-BSA, a control group was injected with vehicle alone, a third group with 2.5 mg of peptide SEQ ID NO:1 or a control peptide. Mice were sacrificed at day 31.

2. Matrigel Sponge Assay

the assay was performed in C57B1 mice as described (3).

To verify whether IL-12 was a mediator of AST function, a neutralizing anti mouse IL-12 antibody (Peproteck Inc. London) and respectively an irrelevant anti phage 13 antibody (5 prime, 3 prime Inc. Boulder, Co. 150 ng/ml) were added to the mixture.

3. Peptide Sequences

Peptide 5 (SEQ ID NO:1): HNRTPENFPCKNL, starting from position 307 of human plasminogen (P00747 Swiss-prot database)

peptide 3: DSSPVSTEQLAPTA from kringle3 right boundary.

peptide 4: SSTSPHRPRFS from kringle1 core.

4. Histology of Matrigel Sponges

After animal sacrifice, pellets were fixed in 4% PAF and paraffin embedded; 4 μM sections were stained with hematoxylin-eosin standard procedure.

5. Chemotaxis

The assay was conducted in 48 wells chambers (Costar Nucleopore, Milan, Italy) according to Falk (4). The lower compartment of each chamber was filled with 27 μL of chemoattractant (IL-8, 50 ng/ml) in RPMI 0.1% BSA (SFM). Serum-free medium (SFM) was used for spontaneous migration without stimulus. The upper compartment was filled with 50 μL of polymorphonucleate (PMN) suspension in SFM (3×10⁶ cells/ml); each experimental point was performed in sextuplicate. Chambers were incubated at 37° C. in 5% CO₂ in humidified atmosphere for 45 minutes. Filters were removed and the cells fixed in 100% ethanol and stained with toluidine blue. The migrated cells were quantified by scanning of the filter surface and by densitometry of blue staining by NIH Imaging Analysis Software.

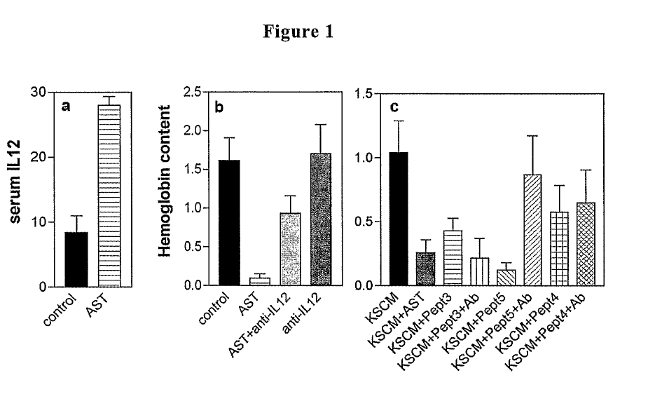

The results are shown in FIGS. 1-3 where:

FIG. 1-a: serum quantification of IL-12 after peri-tumoral treatment with AST. Columns show the geometric means and standard error of the samples after fourt week treatment.

FIG. 1-b: matrigel in vivo angiogenesis assay with AST. Columns show means and standard error of groups comprising six animals each. KS-conditioned medium (KSCM) is the stimulus (control).

FIG. 1-c: matrigel in vivo angiogenesis assay with peptides.

A combination of peptides and anti IL-12 Ab was added to the samples containing KSCM as indicated.

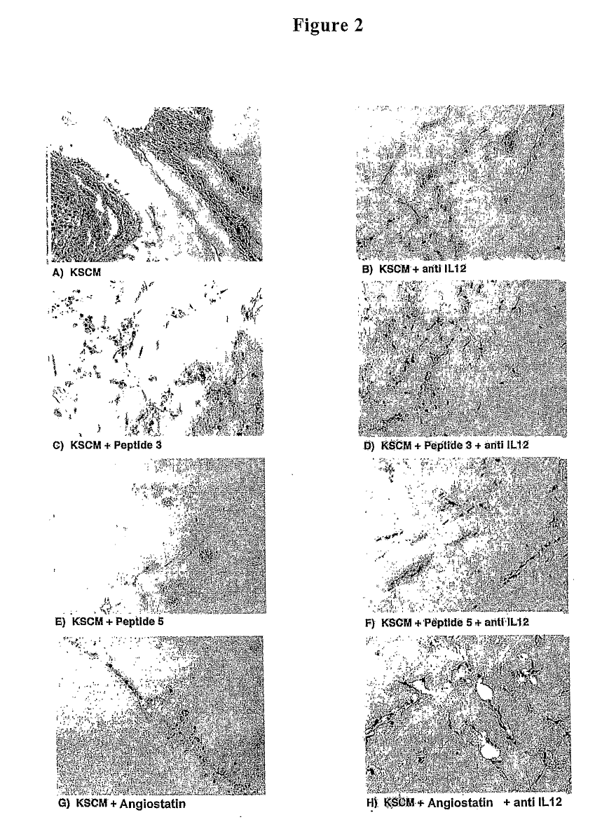

FIG. 2, a-h: standard ematoxylin-eosin staining of the matrigel pellets relative to experiments in FIG. 1-c.

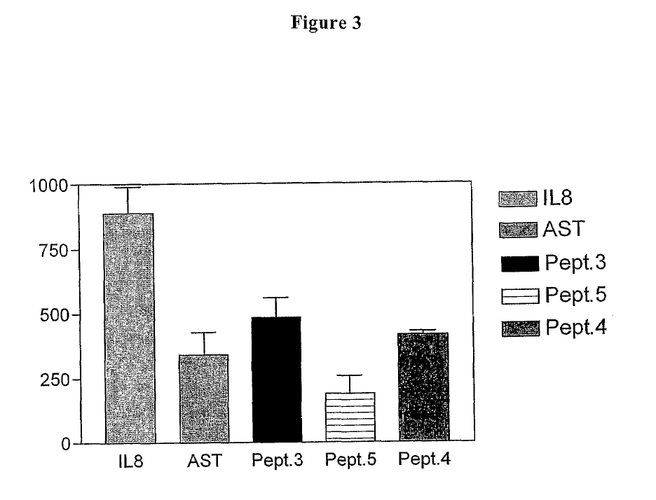

FIG. 3: PMN chemotaxis. Densitometry of migrated cells. IL-8 represents the stimulus in each sample, to which either AST or relevant peptides are added.

### REFERENCE

1. Benelli, R., et Al., FASEB J, 16: 267-269, 2002.
2. M. Schnurr, et Al, J immunol 165, 4704-9 (2000).
3. F. Wilkin, et Al., J immunol 166, 7172-7 (2001).
4. Albini A., et. Al., AIDS. 1994 September; 8(9):1237-44.
5. Falk, W., et Al., J Immunol Methods, 33: 239-247, 1980.

1. Anti-angiogenic peptide having sequence SEQ ID NO:1.

2. Pharmaceutical composition containing the peptide SEQ ID NO:1.

3. Composition according to claim 2, containing an adjuvant stimulating the immune response.

4. The use of peptide SEQ ID NO:1 for the preparation of a medicament for the preventive or therapeutic treatment of diseases or dysfunctions involving or mediated by angiogenesis.

5. The use according to claim 4, where said diseases are selected from tumors, ocular diseases, vascular, cardiovascular and inflammatory diseases, degenerative diseases of organs and tissues, transplant complications, cerebral vascular pathologies.

6. The use according to claim 5, where said diseases are tumors, leukemia, retinopathy, glaucoma, macula degeneration, corneal rejection, retrolenticular fibroplasia, rubeosis, diabetes, rheumathoid arthritis, atherosclerosis, diseases associated to ocular or skin transplants.


SEQUENCE LISTING (3 sequences)

SEQ ID NO: 1 - protein, 13 aa, Homo sapiens
HNRTPENFPCKNL

SEQ ID NO: 2 - protein, 14 aa, Homo sapiens
DSSPVSTEQLAPTA

SEQ ID NO: 3 - protein, 11 aa, Homo sapiens
SSTSPHRPRFS